(19) United States
(12) Patent Application Publication
(10) Pub. No.: US 2022/0062573 A1
(43) Pub. Date: Mar. 3, 2022

(54) SPEECH VALVE

(71) Applicant: SENKO MEDICAL INSTRUMENT Mfg. Co., Ltd., Tokyo (JP)

(72) Inventors: Mitsuru CHIBA, Tokyo (JP); Hideaki KANAZAWA, Shizuoka (JP)

(73) Assignee: SENKO MEDICAL INSTRUMENT Mfg. Co., Ltd., Tokyo (JP)

(21) Appl. No.: 17/420,893

(22) PCT Filed: Oct. 25, 2019
(86) PCT No.: PCT/JP2019/042025

§ 371 Date: Jul. 6, 2021

(30) Foreign Application Priority Data
Jan. 10, 2019 (JP) 2019-002368

(51) Int. Cl. A61M 16/04

(52) U.S. Cl. CPC A61M 16/0468; A61M 2205/3337

(57) ABSTRACT

A speech valve to be connected to a respiration opening of a tracheostomy tube includes: a passage portion communicating with the respiration opening; a balloon disposed within the passage portion, and configured to deform from a shrink state, in which the passage portion is opened, to a bloat state, in which at least a part of the passage portion is closed so as to inhibit discharging of exhaled air through the passage portion; an automatic pump switching between supplying and stopping in accordance with an operation of an operation unit, and continuously supplying air to the balloon when supplying; and a coupling tube connecting the automatic pump and the balloon.

### CROSS-REFERENCE TO RELATED PATENT APPLICATIONS

This application is a U.S. National Phase Application under 35 U.S.C. § 371 of International Patent Application No. PCT/JP2019/042025 filed on Oct. 25, 2019 and claims the benefit of priority to Japanese Patent Application No. 2019-002368 filed Jan. 10, 2019, the contents of both of which are incorporated herein by reference in their entireties. The International Application was published in Japanese on Jul. 16, 2020 as International Publication No. WO2020/144914 under PCT Article 21(2).

### FIELD OF THE INVENTION

The present invention relates to a speech valve for enabling a patient to utter a speech sound.

### BACKGROUND OF THE INVENTION

JP2016-140433A describes a speech valve which is applied to a respiration opening of a tracheostomy tube and includes a balloon capable of closing a passage portion leading to the respiration opening. The speech valve includes a manual pump that accepts a pushing operation by a patient and changes the deflated balloon from a shrink state to a bloat state to close the passage portion.

### CITATION LIST

### Patent Literature

Patent Literature 1: JP2016-140433A

### Technical Problem

In the speech valve described in JP2016-140433A, it is difficult for a patient having a weak grip to operate the manual pump. In addition, repeated pushing of a plunger of the manual pump can cause a lubricant in a syringe of the manual pump to peel off. If the lubricant peels off, the resistance at the time of pushing-in operation of the plunger increases, so that the operation becomes more difficult for a patient having a weak grip. Also, the increased resistance to the spring that pushes the plunger back may cause the balloon to fail to shrink.

### SUMMARY OF THE INVENTION

### Solution to Problem

A speech valve according to one aspect of the present invention is a speech valve to be connected to a respiration opening of a tracheostomy tube, the speech valve comprising: a passage portion communicating with the respiration opening; a balloon disposed within the passage portion, and configured to deform from a shrink state, in which the passage portion is opened, to a bloat state, in which at least a part of the passage portion is closed so as to inhibit discharging of exhaled air through the passage portion; an automatic pump switching between supplying and stopping in accordance with an operation of an operation unit, and continuously supplying air to the balloon when supplying; and a coupling tube connecting the automatic pump and the balloon.

The automatic pump has a pressure limiter limiting an upper pressure limit in the balloon.

The automatic pump has a safety valve opening a pneumatic circuit between the automatic pump and the balloon when not supplying.

A release opening, through which air in the balloon is discharged to an outside, is formed in at least one of the balloon and the coupling tube.

Because the automatic pump is provided for automatically supplying air to the balloon, a patient does not need to perform a manual pushing operation of a manual pump. Thus, the speech valve can be easily operated.

### BRIEF DESCRIPTION OF THE DRAWINGS

FIG. 1 is a schematic side view of a tracheostomy tube to which a speech valve is connected.

FIG. 2 is a schematic exploded perspective view of the speech valve.

FIGS. 3A and 3B are schematic views useful to describe deformations of a balloon. Specifically, FIG. 3A shows the balloon in a shrink state, and FIG. 3B shows the balloon in a bloat state.

FIG. 4 is a schematic perspective view of an automatic pump according to a first embodiment when viewed from the top.

FIG. 5 is a schematic perspective view of the automatic pump according to the first embodiment when viewed from the bottom.

FIGS. 6A and 6B are schematic perspective views of an automatic pump according to a second embodiment. Specifically. FIG. 6A shows a movable cover before sliding, and FIG. 6B shows the movable cover after sliding.

FIG. 7 is a schematic side view of a main body portion according to a modified embodiment.

### DETAILED DESCRIPTION OF THE INVENTION

Illustrative embodiments for carrying out the present invention will be described in detail below with reference to the drawings. The dimensions, materials, shapes, and relative positions of the components described in the following embodiments may be arbitrarily set, and may be changed according to the configuration or various conditions of a device (apparatus) to which the present invention is applied. In addition, unless otherwise specified, the scope of the present invention is not limited to the embodiments specifically described in the following description.

### First Embodiment

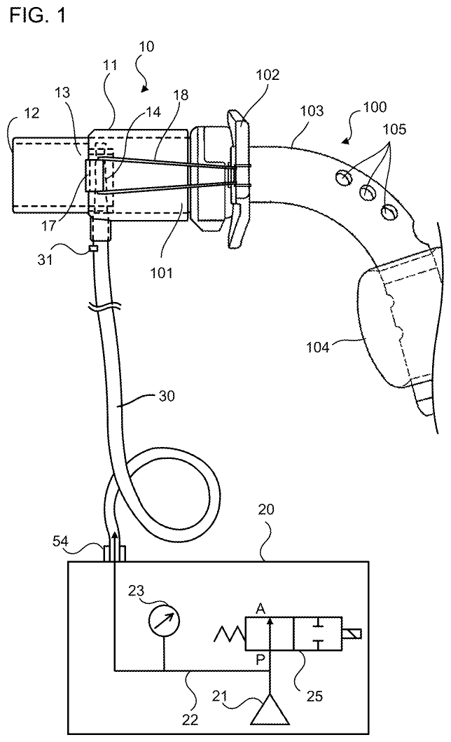

Referring to FIG. 1 to FIGS. 3A and 3B, a speech valve 10 will be described. As shown in FIG. 1, the speech valve 10 is connected to a respiration opening 101 of a tracheostomy tube 100. The respiration opening 101 has a diameter that allows a female connector of a general artificial respiratory circuit to be inserted into a connector portion of the tracheotomy tube 100. The open end of the main body portion 11 of the speech valve 10 on the patient side is a female connector that can be connected to the respiration opening 101 of the main body portion. The open end 12 of the main body portion 11 on the machine side is a male connector having an outer diameter equivalent to that of the respiration opening 101. Thus, the artificial respiratory circuit can be connected to the open end 12 of the main body portion 11 while the main body 11 is being connected to the respiration opening 101.

The tracheostomy tube 100 has a frame 102 for fixing to a throat portion of the intubated patient with a tape (not shown), and a pipe portion 103 to be inserted into a trachea of the patient. The frame 102 extends outwardly from the pipe portion 103, and an opening for winding the tape is formed at each end of the frame 102. A plurality of side holes 105 are formed in the pipe portion 103. Each of the side holes 105 penetrates the inside and the outside of the pipe portion 103 so as to guide exhaled air to a vocal cord side of the patient when the respiration opening 101 is closed by the speech valve 10 (i.e., a balloon 14, which will be described later). If unnecessary, the side holes 105 may not be formed. Below the side holes 105, provided is a sealing portion 104. The sealing portion 104 causes the pipe portion 103 to tightly contact the inside of the trachea of the patient, and prevents air leakage from the outer circumference of the pipe portion 103.

The main body portion 11 of the speech valve 10 has a passage portion 13 that communicates with the respiration opening 101 when connected to the respiration opening 101 of the tracheostomy tube 100. The speech valve 10 also includes a balloon 14 disposed in the passage portion 13. The balloon 14 deforms from a deflated state (shrink state) in which the passage portion 13 is opened to an inflated state (bloat state) in which at least a part of the passage portion 13 is closed so as to inhibit discharging of exhaled air through the passage portion 13. That is, the balloon 14 is operable between an open state in which the passage portion 13 is opened and a closed state in which the passage portion 13 is closed. In addition, the speech valve 10 has an automatic pump 20 which switches between the supply of the air and the stop of the air in accordance with the operation of an operation unit which will be described later. The automatic pump 20 continuously supplies the air to the balloon 14 when the automatic pump supplies the air. That is, the automatic pump 20 automatically supplies the air to the balloon 14 while the patient operates the operation unit. The operation unit is operated by the patient at a position away from the main body portion 11. While the patient is operating the operation unit, the air is continuously supplied to the balloon 14. The speech valve 10 also includes a coupling tube 30 that couples the automatic pump 20 with the balloon 14 so that the air can move between the automatic pump and the balloon. The air supplied from the automatic pump 20 passes through the coupling tube 30.

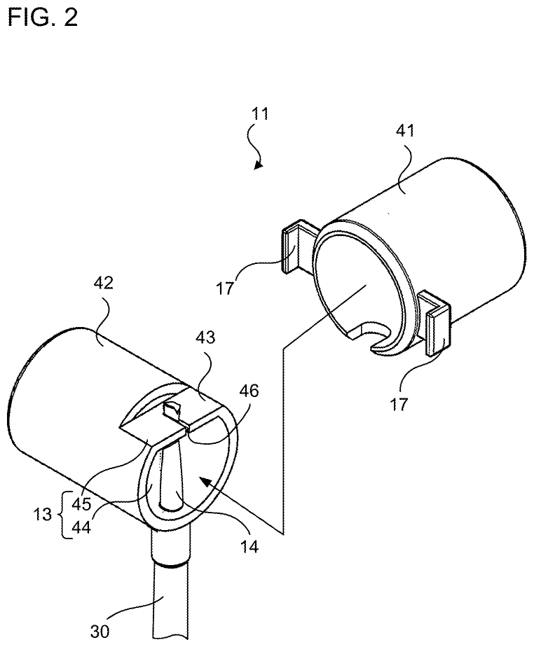

The coupling tube 30 has a sufficient length so that the main body portion 11 is not pulled even when an arm of the patient moves due to convulsion or the like of the patient. In order to prevent the main body portion 11 from easily detaching from the tracheotomy tube 100, protrusions 17 for preventing detachment are formed on both sides of the main body portion 11. As shown in FIG. 2, the protrusions 17 protrude outward on both sides of the main body portion 11, respectively. As shown in FIG. 1, rubber bands 18 are attached to the frame 102. One end of each rubber band 18 passes through an opening of the frame 102. The other end of each rubber band 18 is caught by the associated projection 17 in a state in which the main body portion 11 is attached to the tracheostomy tube 100. Thus, the main body portion 11 is fixed to the tracheostomy tube 100 such that the main body portion 11 is not detached from the tracheostomy tube 100. Even if the speech valve 10 is detached, there is no problem except for the inability to speak. Thus, the fixing by the rubber bands 18 may be omitted.

As shown in FIG. 2, the main body portion 11 of the speech valve 10 includes a female member 41 connected to the respiration opening 101 of the tracheostomy tube 100 and a male member 42 to which the balloon 14 is attached. The female member 41 and the male member 42 are connected to each other. The passage portion 13 is formed by fitting the inner periphery of the female member 41 over the outer periphery of the male member 42. The passage portion 13 is partitioned into a main region 44 and a ventilation region 45 that is narrower than the main region 44 by a partition portion 43 provided in the male member 42. A groove portion 46 for fixing one end of the balloon 14 is formed in the partition portion 43. As the one end of the balloon 14 fits into the groove 46, the one end of the balloon 14 is closed. Thus, the balloon 14 is disposed in the main region 44 with one end thereof being fixed. The balloon 14 in the shrink state has a cylindrical shape. The other end of the balloon 14 serves as a supply port to which air is supplied.

Even if the balloon 14 changes to the bloat state, one end of the balloon 14 is maintained in the shrink state in the ventilation region 45. Therefore, the passage portion 13 is not completely closed, and the breathability is ensured by the ventilation region 45 partitioned by the partition portion 43, so that the safety is enhanced. The size of the ventilation region 45 is appropriately set within a range that does not hinder the utterance. Alternatively, the inner wall of the passage portion 13 may be provided with a protrusion protruding toward the center of the passage portion 13 from the inner wall of the passage portion 13. The protrusion can avoid the close contact between the balloon 14 and the inner wall of the passage portion 13 even when the balloon 14 changes to the bloat state. As a result, even if the balloon 14 changes to the bloat state, a flow path is formed between the inner wall of the passage portion 13 and the balloon 14. Further, the degree of inflation of the balloon 14 may be adjusted by limiting an amount of air supplied from the automatic pump 20. In other words, the balloon 14 may be inflated to such an extent that the balloon 14 and the inner wall of the passage portion 13 do not come into close contact with each other. As a result, even if the balloon 14 changes to the bloat state, a flow path is formed between the inner wall of the passage portion 13 and the balloon 14.

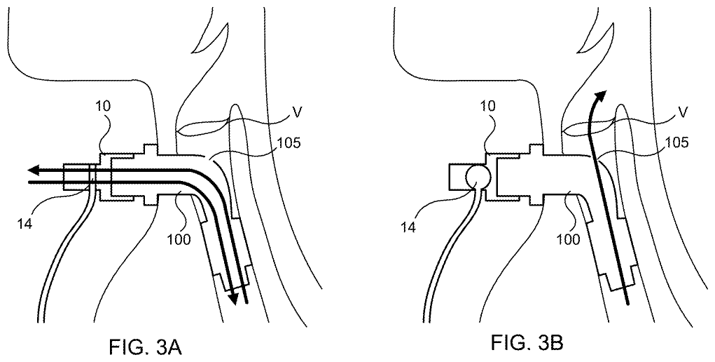

As the air is supplied to the balloon 14 from the automatic pump 20, the balloon 14 deforms from the shrink state shown in FIG. 3A to the bloat state shown in FIG. 3B. As a result, the balloon 14 shifts from the open state in which the passage portion 13 is opened to the closed state in which the passage portion 13 is closed. The balloon 14 closes at least a portion of the passage portion 13 upon bloating (inflating) to inhibit the discharging of exhaled air from the passage portion 13. As a result, the exhaled air of the patient flows toward the vocal cord V through the side holes 105 of the tracheostomy tube 100 without entering the passage portion 13.

The patient may manipulate the automatic pump 20, which is located away from the main body portion 11, and operate the balloon 14 to close the passage portion 13. Therefore, even a patient who has difficulty in blocking the opening in throat with fingers can speak out. On the other hand, in the open state in which the passage portion 13 is opened, the balloon 14 does not hinder the entry of air into the passage portion 13. Therefore, the outside air is sucked into the tracheostomy tube 100 through the passage portion 13. The exhaled air of the patient is discharged to the outside of the patient's body through the passage portion 13.

As shown in FIG. 1, the small automatic pump 20 includes a gas pressure source 21 for supplying the air to the balloon 14, and a pressure gauge 23 for monitoring the gas pressure in a pneumatic circuit 22. Additionally, the automatic pump 20 includes a safety valve 25 which forces the pneumatic circuit 22 in the automatic pump 20 to open when not in operation. In one example, the gas pressure source 21 is an electrically operated pressurizing pump and is driven by electric power supplied from a battery (not shown). Alternatively, the gas pressure source 21 may be a gas cylinder filled with compressed air. Alternatively, the gas pressure source 21 may be a gas cylinder or a compressed air supply device disposed in a patient room, or a gas pipe disposed in the patient room.

The pneumatic circuit 22 extending from the gas pressure source 21 branches into a flow path that extends to the safety valve 25 and another flow path that extends toward the balloon 14. The pressure gauge 23 is connected in the pneumatic circuit 22 and functions as a pressure limiter to control the pressure in the balloon 14 and the pneumatic circuit 22 to be constant. In one example, the pressure gauge 23 detects, with a limit switch or the like, a state in which the tube functioning as the pneumatic circuit 22 is expanded by the internal pressure. The pressure gauge 23 may also detect the pressure in the pneumatic circuit 22 to control the operation of the automatic pump 20. Thus, it is possible to prevent the pressure in the balloon 14 from exceeding the upper limit pressure and avoid the damaging to the balloon 14. Alternatively, the pressure gauge 23 may be a relief valve. The safety valve 25 is, for example, a normally open two-way single-acting solenoid valve, and opens a flow path from the supply port P to the port A while no electricity being supplied. Thus, the pneumatic circuit 22 is opened to the outside at the time of non-operation, and the air is discharged through the safety valve 25.

The coupling tube 30 connects the supply port of the balloon 14 to a discharge port 54 of the automatic pump 20. Further, a pinhole is formed in the coupling tube 30 such that the pinhole functions as a release port 31 for discharging the air from the balloon 14 to the outside in order to prevent the balloon 14 from non-shrinking upon the kink (blockade). The release port 31 is formed in the vicinity of the balloon 14. As the balloon 14 shrinks with its own elastic force, the air in the balloon 14 is continuously discharged to the outside through the release port 31 in a very small amount. Thus, even if the kink occurs in the coupling tube 30, the balloon 14 shrinks to open the passage portion 13. On the other hand, in a normal state in which no kink occurs, air supplied from the automatic pump 20 is automatically supplemented to the balloon 14 in a closed state in which the passage portion 13 is closed. That is, the size of the release port 31 is set such that an amount of air discharged through the release port 31 is smaller than an amount of air supplied from the automatic pump 20. Therefore, the balloon 14 can maintain a constant bulge.

In one example, the release port 31 is formed on the open end 12 side in the coupling tube 30 such that the release port 31 is formed on the side opposite to the side facing the patient. The release port 31 is formed at a position where an unintentional kink does not occur in the coupling tube 30. In other words, the release port 31 is formed immediately below the balloon 14. Alternatively, the release port 31 may be formed in the balloon 14. Alternatively, the release port 31 may be formed in the coupling tube 30 on the tracheostomy tube 100 side, or may be formed on the side portion of the coupling tube 30. The number and size of the release port(s) 31 can be arbitrarily set.

When the patient stops the operation and the speech valve is not in operation (in a no-supply state), the automatic pump 20 is completely stopped and the safety valve 25 is brought into the open state, so that the air in the balloon 14 is immediately discharged through the safety valve 25. As a result, the supply of the air from the automatic pump 20 to the balloon 14 stops. Therefore, the balloon 14 in the bloat state shifts to the shrink state to open the passage portion 13. That is, in addition to the provision of the release port 31, the safety valve 25 is opened when the patient stops the operation. Thus, it is possible to securely cause the balloon 14 to shrink. Therefore, it is possible to suppress the passage portion 13 from being maintained in a closed state. Incidentally, since the air is discharged from the release port 31 in the no-supply state, the safety valve 25 may be omitted.

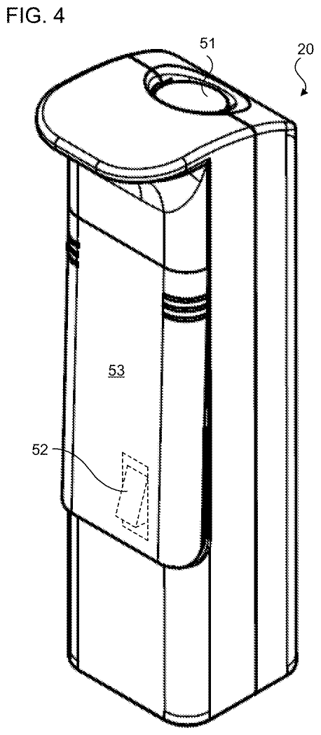

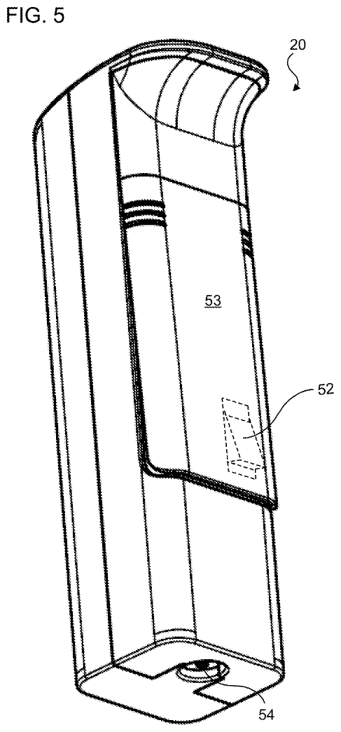

Referring to FIG. 4 and FIG. 5, the operation unit of the automatic pump 20 will be described. In the first embodiment, the operation unit is constituted by a push-button switch 51, which can be pressed, and a limit switch 52 covered with a movable cover 53. Further, the movable cover 53 is movable in a direction of pressing the limit switch 52. For convenience of explanation, the limit switch 52 covered with the movable cover 53 is indicated by a dotted line in FIG. 4 and FIG. 5.

As shown in FIG. 4, the substantially rectangular parallelepiped-shaped automatic pump 20 has the push button switch 51 and the limit switch 52 which function as the operation unit. The button switch 51 can be pressed and operated by the patient pressing down with the first finger. The button switch 51 is exposed on the top surface of the automatic pump 20. When the patient stops the operation by releasing the finger, the button switch is biased by a spring (not shown) and is caused to return to the original position.

The limit switch 52 can be pushed and operated when the patient grips the limit switch 52 with the second finger to the fifth finger of the patient. The limit switch 52 is covered with the movable cover 53. When the patient operates the limit switch 52, the operation by the button switch 51 is validated. That is, when the patient does not operate the limit switch 52, the automatic pump 20 is not driven even if the button switch 51 is operated. Thus, for example, even when the button switch 51 is pressed by unintentional switch operation while the patient is not holding the automatic pump 20, it is possible to prevent the passage portion 13 of the speech valve 10 from being closed.

The limit switch 52 is disposed on a portion on the bottom surface side of the automatic pump 20. The movable cover 53 can move about the axis of rotation at the upper end of the movable cover 53 in a direction approaching the limit switch 52 and in a direction away from the limit switch 52. In one example, the movable cover 53 pushes the limit switch 52 and enables the operation of the button switch 51 as the patient grips the automatic pump 20 with the patient's hand. When the patient releases the hand, the movable cover 53 and the limit switch 52 are biased by the spring (not shown) to return to the original positions, respectively. That is, the movable cover 53 moves in a direction away from the limit switch 52, and the limit switch 52 returns to the original position. Therefore, the operation by the button switch 51 is disabled, and the automatic pump 20 is not driven even if the button switch 51 is operated.

As shown in FIG. 5, the discharge port 54 is formed on the bottom surface of the automatic pump 20, i.e., the surface opposite to the top surface on which the button switch 51 is disposed. The discharge port 54 receives one end of the coupling tube 30, thereby connecting the coupling tube 30 to the automatic pump 20. Further, the top surface of the automatic pump 20 is larger than the bottom surface thereof, and a portion of the top surface protrudes. This makes it easier for the patient to grip the automatic pump 20, so that the patient can operate the limit switch 52 even if the patient has a weak force. Incidentally, the automatic pump 20 has, for example, an LED lamp (not shown) that serves as an operation confirmation lamp covered with the movable cover 53. In operation, the lamp lights up, and it can be visually confirmed that the automatic pump is being operated. Therefore, the movable cover 53 is formed of a light-transmitting material.

In order to operate the automatic pump 20, the speech valve 10 is first connected to the respiration opening 101 of the tracheostomy tube 100. Subsequently, the patient grasps the automatic pump 20 by the patient's hand and pushes the limit switch 52 through the movable cover 53. Next, the patient depresses the button switch 51 while depressing the limit switch 52. When the patient is performing the above-described predetermined operations, the automatic pump 20 is driven and the air is supplied. The air discharged from the discharge port 54 is supplied to the balloon 14 via the coupling tube 30. At this time, since an amount of air discharged through the release port 31 is smaller than an amount of air supplied from the automatic pump 20, the balloon 14 in the shrink state changes to the bloat state. Then, the balloon 14 closes the passage portion 13 to allow the patient to utter a speech sound.

When the patient wants to finish the speech utterance, the patient stops the operation of the button switch 51 and releases the finger from the button switch 51. As a result, the button switch 51 returns to the original position such that driving of the automatic pump 20 is stopped, and the safety valve 25 is opened. Therefore, the supply of the air to the balloon 14 stops. The air between the automatic pump 20 and the balloon 14 is discharged to the outside through the safety valve 25 and the release port 31.

According to the speech valve 10 of the above-described first embodiment, the balloon 14 can be easily operated by the automatic pump 20 even if a patient has a weak grip. In addition, the safety valve 25 and the release port 31 prevent the balloon 14 from no-shrinking when not in operation. That is, the safety valve 25 closes the pneumatic circuit 22 only during operation whereas the pneumatic circuit 22 is forcibly opened during non-operation. As a result, in no-electricity supply time, i.e., when the patient does not operate the speech valve, the air can be reliably extracted from the balloon 14. Further, even when the coupling tube 30 is occluded, the air in the balloon 14 is discharged through the release port 31. Therefore, it is possible to reliably cause the balloon 14 to shrink. Further, because the limit switch 52 is provided, it is possible to prevent erroneous operation.

### Second Embodiment

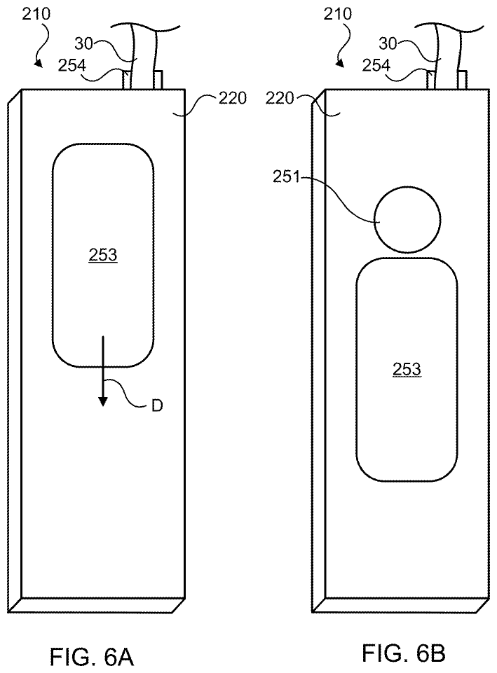

A second embodiment will be described with reference to FIG. 6A and FIG. 6B. The second embodiment differs from the first embodiment in that the automatic pump 220 includes a movable cover 253 which is slidable between a first position (FIG. 6B) for exposing the operation unit and a second position (FIG. 6A) for covering the operation unit. In the description of the second embodiment, differences from the first embodiment will be described, the components already described are denoted by the same reference numerals, and description thereof is omitted. Unless otherwise mentioned, the components denoted by the same reference numerals perform substantially the same operations and functions, and their effects and advantages are substantially the same.

The movable cover 253 is slidable in the sliding direction D pointing downward in FIG. 6A. For example, when the patient slides the movable cover 253 with a finger, the movable cover 253 moves to the first position in a direction away from the button switch 251 that serves as the operation unit. When the movable cover 253 slides to the first position, as shown in FIG. 6B, the button switch 251 is exposed from the movable cover 253. That is, the movable cover 253 is in the second position covering the button switch 251 at the time of non-operation, and can move from the second position to the first position for exposing the operation unit. The patient can then operate the exposed button switch 251. When the patient releases the finger to stop the operation, the button switch 251 is biased by a spring (not shown) and returns to the original position.

In order to operate the automatic pump 220, the speech valve 210 is first connected to the respiration opening 101 of the tracheostomy tube 100. Subsequently, the patient slides the movable cover 253 to expose the button switch 251. Next, the patient depresses the exposed button switch 251. When the patient is performing the above-described predetermined operations, the automatic pump 220 is driven and the air is supplied. The air discharged from the discharge port 254 is supplied to the balloon 14 via the coupling tube 30. At this time, since an amount of air discharged through the release port 31 is smaller than an amount of air supplied from the automatic pump 220, the balloon 14 in the shrink state changes to the bloat state. Then, the balloon 14 closes the passage portion 13 to allow the patient to utter a speech sound.

When the patient wants to finish the speech utterance, the patient stops operating the button switch 251 and releases the finger from the button switch 251. As a result, the button switch 251 returns to the original position such that the driving of the automatic pump 220 is stopped, and the safety valve 25 is opened. Therefore, the supply of the air to the balloon 14 stops. The air between the automatic pump 220 and the balloon 14 is discharged to the outside through the safety valve 25 and the release port 31. Further, when the patient releases the finger from the movable cover 253, the movable cover 253 is caused to return to the original position by a spring (not shown). Therefore, the button switch 251 is covered with the movable cover 253.

According to the speech valve 210 of the above-described second embodiment, the balloon 14 can be easily operated by the automatic pump 220 even if a patient has a weak grip. In addition, the safety valve 25 and the release port 31 prevent the balloon 14 from no-shrinking when not in operation. That is, the pneumatic circuit 22 is forcibly opened when not in operation. Thereby, in no-electricity supply time in which the patient does not operate, the air can be reliably extracted from the balloon 14. Further, even when the coupling tube 30 is occluded, the air in the balloon 14 is discharged through the release port 31. Therefore, it is possible to reliably cause the balloon 14 to shrink. Further, when not in operation, the button switch 251 is covered with the movable cover 253. Thus, for example, it is possible to prevent unintentional switch operation while the patient is not holding the automatic pump 220.

While the present invention has been described with reference to the respective embodiments, the present invention is not limited to the above-described embodiments. Also included in the present invention are inventions which are modified and equivalent to the present invention within a range not inconsistent with the present invention. In addition, each of the embodiments and each modification can be appropriately combined within a range not contrary to the present invention.

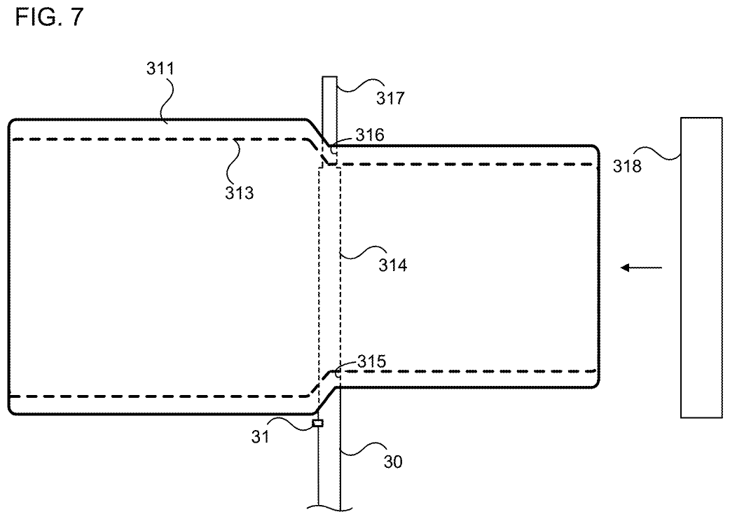

For example, as illustrated in FIG. 7, a main body portion 311 of the speech valve 10 may be integrally formed. In this configuration, since the partition portion 43 is not formed in the main body portion 311, the ventilation region 45 is not formed. This prevents exhaled air from leaking out of the ventilation region 45 when the balloon 314 prevents the exhaled air from being discharged through the passage portion 313. Accordingly, it becomes easier for a patient to utter a speech sound. Further, since the balloon 314 is caused to shrink by the release port 31 and air permeability is ensured, safety is not deteriorated.

In one example, opposite ends of the balloon 314 are inserted into a large circular portion 315 and a small circular portion 316 formed in the main body portion 311, respectively. An adhesive is applied to a portion of the balloon 314 protruding from the main body portion 311 and a portion of the coupling tube 30 protruding from the main body portion 311, and are adhered to the reinforcing ring 318 which is fitted over the main body portion 311. Thus, the balloon 314 is fixed to the main body portion 311. Further, an adhesive is filled inside the protruding portion 317 of the balloon 314 protruding from the main body portion 311. By means of the adhesive filled inside the protrusion portion 317 and the reinforcing ring 318, the protruding portion 317 of the balloon 314 is closed. Incidentally, the reinforcing ring 318 is partially opened (not shown) so that the connecting portion between the coupling tube 30 and the balloon 314 is not closed.

The release port 31 may be formed in each of the coupling tube 30 and the balloon 14. In the case where the erroneous operation does not cause a problem, the movable covers 53 and 253 may be omitted. It should be noted, however, that each of the movable covers 53 and 253 provides the effect of the double switch or switch cover, and therefore it is possible to reliably prevent erroneous operation of the switch. Also, the operation unit of each of the automatic pumps 20 and 220 is not limited to a switch. For example, each of the automatic pumps 20 and 220 may have a touch panel as the operation unit. In such configuration, the patient touches soft buttons displayed on the touch panel to perform the operation. The touch panel can be covered with a movable cover to prevent erroneous operation. Further, each of the automatic pumps 20 and 220 may have an operation unit wirelessly connected thereto or wire-connected thereto.

Part or all of the above-described embodiments may also be described as in the following supplementary notes, but are not limited to the following.

### (Supplementary Note 1)

A speech valve to be connected to a respiration opening of a tracheostomy tube, the speech valve comprising:

a passage portion communicating with the respiration opening;

a balloon disposed within the passage portion, and configured to deform from a shrink state, in which the passage portion is opened, to a bloat state, in which at least a part of the passage portion is closed so as to inhibit discharging of exhaled air through the passage portion;

an automatic pump switching between supplying and stopping in accordance with an operation of an operation unit, and continuously supplying air to the balloon when supplying; and

a coupling tube connecting the automatic pump and the balloon,

wherein the operating unit of the automatic pump can be pushed in by a pushing operation, and returns to an original position when the pushing operation is released.

### (Supplementary Note 2)

The speech valve according to Supplementary Note 1, wherein the operation unit is covered with a movable cover.

### (Supplementary Note 3)

The speech valve according to Supplementary Note 2, wherein the movable cover is movable in a direction of pushing the operation unit.

### (Supplementary Note 4)

The speech valve according to Supplementary Note 2, wherein the movable cover is slidable between a first position where the operation unit is exposed and a second position where the operation unit is covered.

### REFERENCE SIGNS LIST

10: Speech valve
13: Passage portion
14: Balloon
20: Automatic pump
22: Pneumatic circuit
23: Pressure gauge (pressure limiter)
25: Safety valve
30: Coupling tube
31: Release port
51: Button switch (operation unit)
52: Limit switch (operation unit)
100: Tracheostomy tube
101: Respiration opening
210: Speech valve
220: Automatic pump
251: Button switch (operation unit)
313: Passage portion
314: Balloon

1. A speech valve to be connected to a respiration opening of a tracheostomy tube, the speech valve comprising:
a passage portion communicating with the respiration opening;
a balloon disposed within the passage portion, and configured to deform from a shrink state, in which the passage portion is opened, to a bloat state, in which at least a part of the passage portion is closed so as to inhibit discharging of exhaled air through the passage portion;
an automatic pump switching between supplying and stopping in accordance with an operation of an operation unit, and continuously supplying air to the balloon when supplying; and
a coupling tube connecting the automatic pump and the balloon.

2. The speech valve according to claim 1, wherein the automatic pump has a pressure limiter limiting an upper pressure limit in the balloon.

3. The speech valve according to claim 1, wherein the automatic pump has a safety valve through which air between the automatic pump and the balloon is discharged when not supplying.

4. The speech valve according to claim 1, wherein a release opening, through which air in the balloon is discharged to an outside, is formed in at least one of the balloon and the coupling tube.
